(19) United States
(12) Patent Application Publication
(10) Pub. No.: US 2006/0096858 A1
(43) Pub. Date: May 11, 2006

(54) Potentiometric urea sensor based on ion-selective electrode

(75) Inventors: Shen-Kan Hsiung, Jungli City (TW); Jung-Chuan Chou, Douliou City (TW); Tai-Ping Sun, Jhongli City (TW); Chung-We Pan, Wandan Township (TW); Nien-Hsuan Chou, Jhongli City (TW)

Correspondence Address: APEX JURIS, PLLC;TRACY M HEIMS, LAKE CITY CENTER, SUITE 410, 12360 LAKE CITY WAY NORTHEAST, SEATTLE, WA, 98125, US

(73) Assignee: Chung Yuan Christian University, Jungli City (TW)

(21) Appl. No.: 10/984,495

(22) Filed: Nov. 8, 2004

(51) Int. Cl. G01N 33/487; C12Q 1/00

(52) U.S. Cl. 204/403.05; 204/403.01

(57) ABSTRACT

The present invention is a sensor for detecting urea using a separating-style structure of ion-selective electrode, in which an ammonium ion-selective membrane is immobilized on a conductive layer on the surface of a substrate, said conductive layer has a sensing region and a non-sensing region after packaged, and a conductive line is used to retrieve a sensing signal from said conductive layer. In the end, employing the enzyme immobilization method to immobilize a urea enzyme onto ammonium ion-selective membrane, thus the fabrication of a potentiometric urea sensor is completed.

### BACKGROUND OF THE INVENTION

1. Field of the Invention

The present invention relates to a potentiometric urea sensor, particularly to a potentiometric sensor which using an ammonium ion-selective electrode as the base to reduce the effect of temperature and light on the device and can integrate a semiconductor process for the production thereof.

2. Description of the Prior Art

In recent years, since electronic technologies are rising and flourishing, the technology of bio-device has been further applied to the design of a sensor. In the clinical examination of a hospital/clinic, the blood urea nitrogen (BUN) is a primary indictor for detecting the kidney failure of a human body, may suffer from the chronic rental failure once BUN over 20 mg/dl and increasing continuously. Alternatively, the examination of urea not only can response the intake and catabolism of protein, but also has closely relations with the functions of the kidney, the liver, and the adrenal gland endocrine. Thus, it is advantageous to develop a bio-sensor capable of detecting such substances for evaluating the function of a kidney. At present, although the urea concentration can be detected by spectrum analysis directly, the enzyme method still is widely employed.

Related patents and documents are described as following:

(1) U.S. Pat. No. 5,804,047(as cited reference #1) discloses an enzyme sensing system suitable for detecting a specific substance wherein a electrode immobilized the enzyme can immobilize a mixture, which comprising a conductive enzyme and other conductive material formed by using covalent bonds to connect the enzyme and the electron transport substance, and the ways to immobilize a enzyme onto a base material are screen printing, brushing, and the like.

(2) U.S. Pat. No. 5,858,186 (as cited reference #2) discloses an electrochemical sensor for quantitatively detecting the urea concentration using the dialysis waste liquid in the process of blood dialysis. The sensor uses an enzyme to hydrolyze the urea and detects the variation of pH generated by the hydrolysis. The structure used by the sensor can be mass-produced for greatly reducing cost, so the structure is advantageous to be developed as a disposable sensor. For a typical application, this sensor is usually used to diagnose the cancel point of the blood dialysis at an inspection center or collocated with an appropriate computer system. This sensor can also be used by a dialysis patient at home, since it only requires a bit of blood sample to perform detection.

(3) U.S. Pat. No. 5,474,660 (as cited reference #3) discloses an apparatus and a method thereof for detecting ammonium ion concentration, wherein an ammonia gas sensor is placed into a container, and a solution containing ammonium ions is placed into a partial region of the container; hydroxyl ions are generated from the solution by an electrochemical generator at the vicinity of the container placing the ammonia gas sensor, and then the sensor detects the ammonia gas through a film, transformed by the ammonium ions in the solution. The sensor disclosed by this patent thus using the above-mentioned method to detect the ammonium ion concentration in a solution.

(4) U.S. Pat. No. 6,021,339 (as cited reference #4) discloses a uric acid multiple sensor which comprising a sensing device for measuring urea and at least one component for detecting sodium and chlorine ions in uric acid. As far as we know, the specific weight of uric acid is based on the detected signals generated from the concentration of each device. Besides, a component for detecting the units of glucose must be added herein and then finally the particular specific weight in sugar can be used to correct the measured sugar (that is, glucose base line). After that, after all uric acid secreted 24 hours, the detected conditions can be understood simply and accurately from a partial uric acid.

(5) U.S. Pat. No. 4,970,145 (as cited reference #5) discloses an enzyme electrode fabricated using a carbon electrode as the base structure, the enzyme electrode with this structure allows the enzyme (such as glucose oxidized enzyme) attach on the electrode, thus to fabricate an amperometric sensor with good response and stability. The substrate material of the electrode is a thin carbon electrode plated with platinum seldom need to use the formula of electron transport substance and can perform detection with the condition that the dissolved oxygen at low level. The enzyme sensor runs measurement in a 10 mM glucose solution, and the reaction result is a current density having several hundreds microampere/cm² with a short response time. While preserved under a humid environment at room temperature, the sensor still has a good stability and several months of its working life.

(6) U.S. Pat. No. 5,397,451 (as cited reference #6) discloses an amperometric and dry-operated ion-selective electrode which comprising a work electrode and an auxiliary electrode, both are fabricated on an insulating substrate. A first layer is a hydrophilous polymer, but the ion-selective membrane using a non-hydrophilous polymer.

However, the above-described documents and patents mostly are that measuring indirectly the pH value or the ammonia gas transformed by ammonium ions and none of them presented that directly sensing the concentration of the ammonium ions; also not laying emphasis on the utilization of semiconductor process for production, and their enzyme immobilization methods are more complicated. In view of this, after having collected data and designed deliberate experiments for many years, the inventor finally demonstrated the patentability of the potentiometric urea sensor of the present invention.

### SUMMARY OF THE INVENTION

The object the present invention is to provide a potentiometric urea sensor with a flat structure.

The second object of the present invention is to provide a potentiometric urea sensor, using ammonium ion-selective electrode as base, and can integrate a semiconductor process for the production thereof.

The second object of the present invention is to provide a potentiometric urea sensor which can be packaged easily, reduced cost, and reduced the effect of the temperature and light on said sensor.

A potentiometric urea sensor capable of achieving the objects of the invention is fabricated using an ammonium ion-selective electrode as base, suitable for examining the urea concentration in a water solution. The potentiometric urea sensor comprises a insulating or non-insulating substrate on which a non-insulating solid-state ion sensing membrane is deposited for detecting the pH value of a solution; a region is preserved as a sensing window during packaging; a conductive line is fixed on the substrate as a transmission line of the sensing signal, and is packaged with the substrate and the non-insulating solid-state ion sensing membrane; and then an ammonium ion-selective membrane is immobilized on the sensing window of the packaged non-insulating solid-state ion-selective sensing membrane to further form a sensing window of the ammonium ion-selective membrane; a urea enzyme film is immobilized on the sensing window of the ammonium ion-selective membrane to construct a ammonium ion-selective electrode; and, a readout circuit is connected with the conductive line for reading the sensing signal from the ammonium ion-selective electrode.

### BRIEF DESCRIPTION OF THE DRAWINGS

FIG. 1 A-C shows a schematic process for a potentiometric urea sensor.

FIG. 2 shows the measurement circuit diagram of a potentiometric urea sensor.

FIG. 3 is a linear calibration curve of a response potential measured by an ammonium ion-selective electrode while the ammonium concentration ranging from 0.1 mmole/l to 1 mole/l;

FIG. 4 shows the relationship chart between the response potential and time of the potentiometric urea sensor.

FIG. 5 shows a linear calibration curve of a response potential measured by a potentiometric urea sensor in a linear measurement range.

FIG. 6 is a calibration curve of a response potential measured by a potentiometric urea sensor from a solution for measuring urea ranging from 0.8 μmole/l to 10 mmole/l and pH=7.5;

FIG. 7 is a calibration curve of a response potential measured by a potentiometric urea sensor under a measurement environment with different pH values;

FIG. 8 shows the max response potential measured by a potentiometric urea sensor under a measurement environment with different pH values.

### DETAILED DESCRIPTION OF THE PREFERRED EMBODIMENT

(1) Example 1

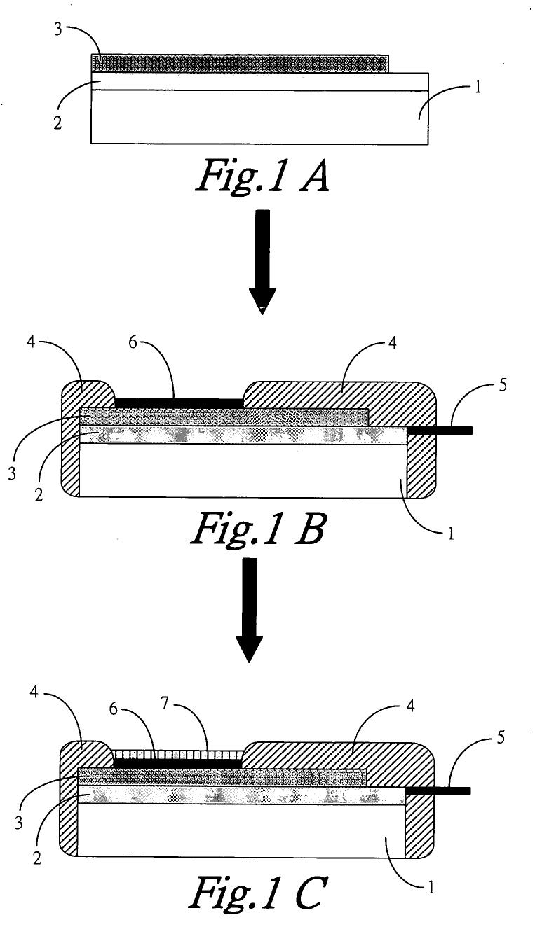

As shown in FIG. 1A, a schematic process for a potentiometric urea sensor made by a ammonium ion-selective electrode, in which a tin dioxide (SnO₂) film 3 with thickness about 2000 Å is sputtered on a substrate containing a glass layer 1 and a tin dioxide layer 2 to entirely form a solid-state pH ion sensing electrode for detecting the pH value in a solution. The above substrate also can be a single layer substrate, such as ceramic substrate, glass substrate, etc, on which a SnO₂ film is sputtered directly and a conductive line is fixed on said SnO₂ film.

As shown in FIG. 1B, using a silver paste, a conductive line 5 is adhered to an end of the above SnO₂ layer 2 uncovered by SnO₂ film 3 as a transmission line of the sensing signal and is packaged by an epoxy resin 4. The packaging also forms a 2×2 mm² sensing window as a sensing region, thus complete the packaging of the solid-state pH ion sensing electrode. After packaging, an ammonium ion-selective membrane 6 is added on the sensing window of the above SnO₂ film by using a mixed solution about 2 μl with following components:

(a1) poly(vinyl chloride) carboxylated, sebacate, DOS: 66% and ammonium ion-selective substance (Nonactin): 1%.

(a2) a conjugate base (Tris(hydroxymethyl)-aminomethane, Tris): 20 mmole/l and a conjugate acid (Ethylen-diaminetetraacetic acid (disodium salt), EDTA): 1.0 mmole/l, the pH value is adjusted to be 7.5 by hydrochloric acid (HCl).

As shown in FIG. 1C, further using a photopolymer, a urea enzyme film 7 is immobilized on the sensing window of the above ammonium ion-selective membrane, wherein the photopolymer is poly(vinyl alcohol)-styrylpyridinium (PVA-SbQ) with components as follows: Poly(vinyl alcohol) Bearing Styrylpyridinium Groups, (degree of polymerization 3500, degree of saponification 88, betaine Sbq 1.05 mol %, solid content 10.22 mol %, pH 5.7, SPP-H-13). Following are the components of the urea enzyme film 7: after diluted with a 125 mg/100 μl, pH=7.0 5 mmole/l phosphate solution, PVA-SbQ mixed with a urea solution (a 10 mg/100 μl, pH 7.0, 5 mmole/l phosphate solution) in the ratio of 1:1.

Upon the operation, the above mixed solution of urea/PVA-SbQ about 10 μl can be fetched and dropped on the ammonium ion-selective membrane 6, and then the sensor can be placed and irradiated with an 4 W ultraviolet light at 365 nm for 20 min. Since the illumination of the above ultraviolet light, which utilize the feature that a photopolymer will be polymerized during ultraviolet light exposure, can immobilize the urea enzyme on the sensing window of the ammonium ion-selective membrane 6, and then complete the fabrication of the urea sensor.

(2) Example 2

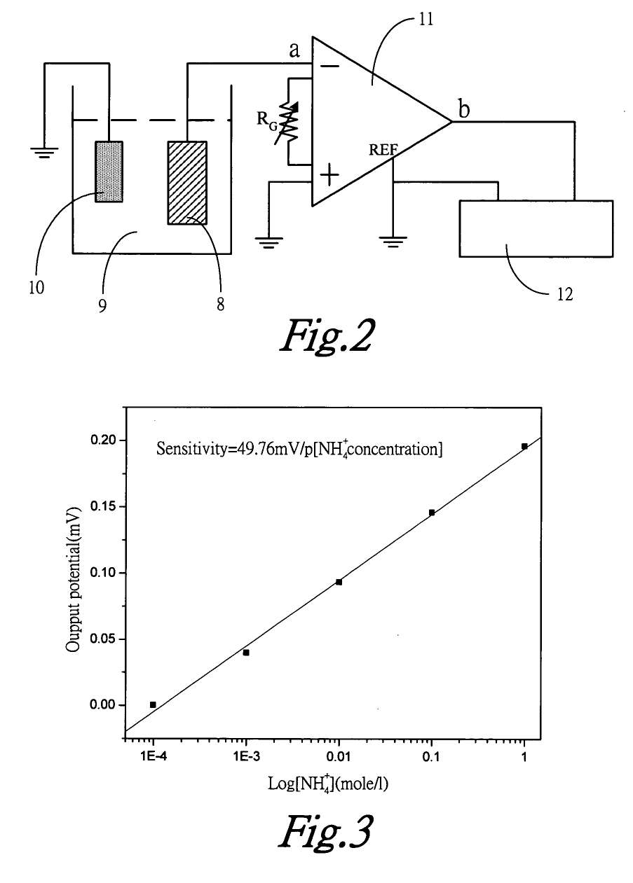

As shown in FIG. 2, a measurement circuit for a potentiometric urea sensor, in which the readout circuit is a instrumentation amplifier 11, the urea sensor 8 placed in a buffer solution 9 for urea measurement is connected to the negative input a of the instrumentation amplifier, while a silver/chloride silver electrode 10 correspondingly provides a reference stable potential, so as to measure the response potential of the sensor. The output end b of the instrumentation amplifier 11 is connected to a multi-function digital meter 12.

The way to use the potentiometric urea sensor includes following steps:

Step 1, using an instrumentation amplifier as the readout circuit of the potentiometric urea sensor;

Step 2, placing and stabilizing the potentiometric urea sensor into a buffer solution before measurement, and using the stabilized response potential as the reference potential;

Step 3, placing the potentiometric urea sensor into a solution to be measured and recording the response potential.

(3) Example 3

FIG. 3 is a linear calibration curve of a response potential, measured by an ammonium ion-selective electrode while the ammonium concentration ranging from 0.1 mmole/l to 1 mole/l, using the measurement circuit shown in FIG. 2. The sensitive characteristic of the ammonium ion-selective electrode is measured while the ammonium concentration ranging from 0.1 mmole/l to 1 mole/l, and via the calculation of the linear calibration curve of the response potential, to ensure the sensitivity of the device locate within a stable range.

(4) Example 4

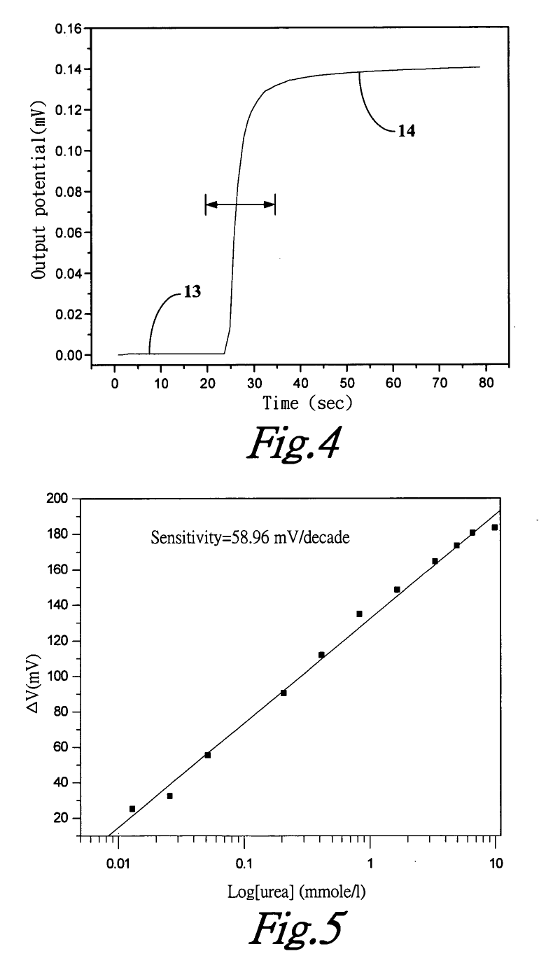

FIG. 4 shows the relationship chart of the response potential and time of the potentiometric urea sensor, using the measurement circuit shown in FIG. 2. First, the urea sensing device is placed into a Tris-HCL buffer solution (20 mmole/l, pH 7.5). After the potential 13 stabilized, using the sensing device to measure the response potential 14 of a solution for measuring enzyme. Seen from the relationship chart, the response potential can reach 90% of the max response potential even the response time less than 15 sec. (about 20 sec. to about 35 sec.).

(5) Example 5

FIG. 5 shows the linear calibration curve of the response potentials are measured by the potentiometric urea sensor with a linear range from 0.02 mmole/l to 1 mmole/l, using the measurement circuit shown in FIG. 2. After calculation with the chart, the sensitivity of the nsor is obtained.

(6) Example 6

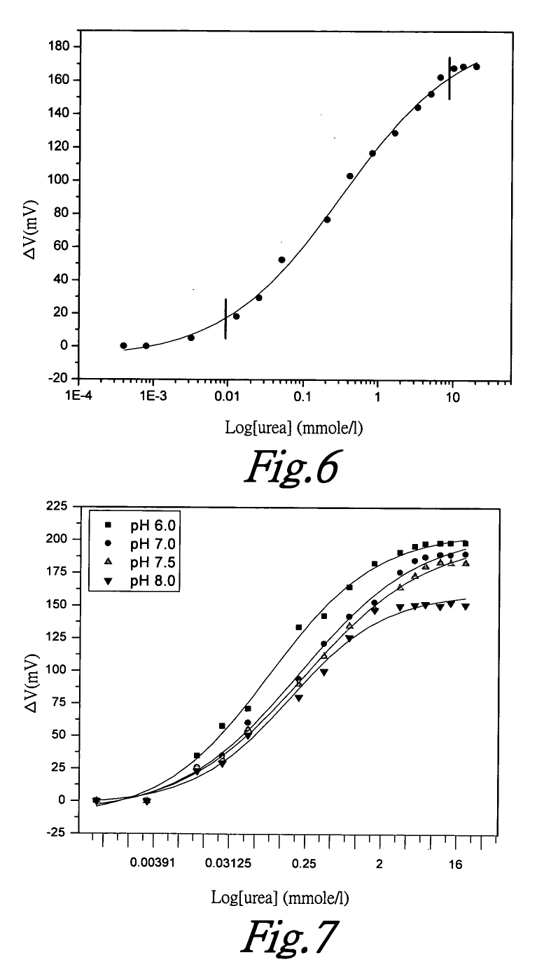

As shown in FIG. 6, the response potential results of a solution for measuring urea with a concentration ranging from 0.8 μmole/l to 10 mmole/l and the pH value 7.5, are measured by the potentiometric urea sensor, using the measurement circuit shown in FIG. 2. Observed from the chart, the linear measurement range of the urea sensing device is from 0.02 mmole/l to 10 mmole/l, minimum measurement is 3 μmole/l, so the linear measurement range can contain the standard urea measurement range of a human body (2.8 mmole/l to 7.12 mmole/l).

(7) Example 7

As shown in FIG. 7, the response potential results of a solution for measuring urea with a concentration ranging from 0.8 μmole/l to 10 mmole/l and with different pH values, are measured by the potentiometric urea sensor, using the measurement circuit shown in FIG. 2. The object is to observe how the pH value of the solution to be measured varies may affect the response potential and the calibration curve. As shown in FIG. 7, the higher pH value of the measured environment, the narrower linear measurement range and the smaller response potential difference.

(8) Example 8

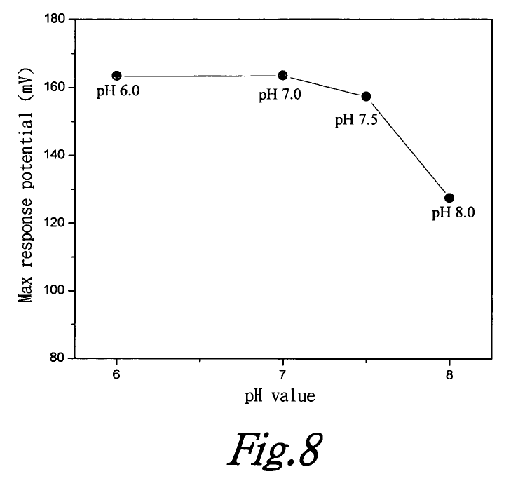

FIG. 8 is a chart of the max response potentials obtained from the measured results in FIG. 7 using solutions to be measured with different pH values and Table 1 lists the values of max response potentials and the linear measurement ranges. As the measured results shown in FIG. 7 and FIG. 8, the device has more stable response potential and measurement range when pH ranging from 6 to 7.5. Considering the factors such as the working environment of an ammonium ion-selective electrode ranging from pH 6.0 to pH 8.0 and the blood pH of a human body ranging from pH 7.35 to pH 7.45, so pH 7.5 is the best response environment of the device.

### Table 1 the Measured Results Obtained From Measured Environments With Different pH, Using the Potentiometric Urea Sensor

As compared with the above-mentioned technology of the cited references, the present invention can provide more characteristics and advantages described as following:

| pH value of the measured environment | pH 6.0 | pH 7.0 | pH 8.0 |
|---|---|---|---|
| max response potential (mV) | 198.067 | 189.78 | 151.09 |
| linear measurement range (mmole/l) | 0.4-10 | 0.4-6.5 | 0.4-5 |

1. As for the enzyme immobilization method, which described in cited reference #1 is screen printing or brushing, but the present invention uses a photopoly-mer to immobilize the urea enzyme.
2. As for a pH sensor, cited reference #2 mainly measures the hydroxyl ions generated from the hydrolysis of ammonium, but the present invention measures the ammonium concentration directly.
3. As for the way to obtain the ammonium concentration, the cited reference #3 using an ammonia gas sensor to measure the ammonia gas transformed by the ammonium ions, but the present invention measures the ammonium concen-tration directly.
4. As for the way to obtain the ammonium concentration, cited reference #4 measuring the sodium and chlorine ions in a urea solution, but the present invention measures the ammonium concentration directly.
5. As for the fabrication base, cited reference #5 using a carbon electrode, but the present invention using an ammonium ion-selective electrode.
6. As for the structure, cited reference #6 is an amperometric and dry-operated ion-selective electrode containing a hydrophilous layer and a non-hydro-philous layer, but the present invention is potentiometric-type without a hydrophilous layer and a non-hydrophilous layer.
7. Except the above-described differences, since the measurement of the present invention can be performed directly, faster and more accurate than cited references, and simpler to be fabricated due to the flat structure thereof.

The above detail description is directed to the embodiments of the present invention, rather than using those examples to limit the scope of the present invention. All equivalents or modifications without departing from the spirit of the present invention should be encompassed in the claimed scope.

To summarize the above description, the present application not only is innovative in technology, but also has the above-mentioned characteristics and advantages. Obviously, the present invention conforms to novelty and non-obviousness that are statutory prerequisites for claiming an invention. Therefore, according to law, an application of this invention is brought up for the approbation.

Many changes and modifications in the above described embodiment if the invention can, of course, be carried out without departing from the scope thereof. Accordingly, to promote the progress in science and the useful arts, the invention is disclosed and is intended to be limited only by the scope of appended claims.

What is claimed is:

1. A potentiometric urea sensor, using an ammonium ion-selective electrode as a fabrication base, suitable for detecting urea concentration in a water solution, said potentiometric urea sensor comprising:
an insulating or a non-insulating substrate, with a non-insulating solid-state ion sensing membrane to detect the pH value of a solution;
a non-insulating solid-state ion sensing membrane, deposited on the substrate, and a preserved region for use as a sensing window during packaging;
a conductive line fixed on the substrate as a transmission line of a sensing signal, packaged with the substrate and the non-insulating solid-state sensing membrane;
an ammonium ion-selective membrane, immobilized on the sensing window of the packaged non-insulating solid-state ion-selective sensing membrane, to further form a sensing window of the ammonium ion-selective membrane;
an urea enzyme film, immobilized on the sensing window of the ammonium ion-selective membrane, to construct an ammonium ion-selective electrode; and
a readout circuit, connected with the conductive line, for reading the sensing signal from the ammonium ion-selective electrode.

2. The potentiometric urea sensor of claim 1, wherein the substrate is a ceramic substrate.

3. The potentiometric urea sensor of claim 1, wherein the substrate is a glass substrate.

4. The potentiometric urea sensor of claim 1, wherein the substrate is composed of indium tin oxide (ITO)/glass.

5. The potentiometric urea sensor of claim 1, wherein the non-insulating solid-state ion sensing membrane is a tin dioxide (SnO₂) film, deposited on the substrate, forming a layered structure of SnO₂/ITO/glass.

6. The potentiometric urea sensor of claim 5, wherein the sputtered thickness of the SnO₂ film is 2000 Å.

7. The potentiometric urea sensor of claim 1, wherein the conductive line is connected to the non-insulating portion of the substrate.

8. The potentiometric urea sensor of claim 1, wherein the material for packaging the substrate and the non-insulating solid-state ion sensing membrane and the conductive line is an epoxy resin.

9. The potentiometric urea sensor of claim 8, wherein a solid-state pH ion sensing electrode can be solely formed after packaging the substrate, the non-insulating solid-state ion sensing membrane and the conductive line.

10. The potentiometric urea sensor of claim 1, wherein the ammonium ion-selective membrane comprises: Poly(vinyl chloride) carboxylated: 33%, dimethyl sebacate: 66%, ammonium ion-selective substance: 1%; conjugate base: 20 mmole/l and conjugate acid: 1.0 mmole/l, the pH value is adjusted to be 7.5 by hydrochloric acid(HCl).

11. The potentiometric urea sensor of claim 1, wherein a photopolymer is utilized to immobilize the urea enzyme film on the ammonium ion-selective membrane.

12. The potentiometric urea sensor of claim 11, wherein the photopolymer is poly(vinyl alcohol)-styrylpyridinium (PVA-SbQ).

13. The potentiometric urea sensor of claim 1, wherein the urea enzyme film comprises:
dilution with a 125 mg/100 μl, pH=7.0 5 mmole/l phosphate solution, PVA-SbQ mixed with a urea solution (10 mg/100 μl, pH 7.0, 5 mmole/l phosphate solution) in the ratio of 1:1.

14. The potentiometric urea sensor of claim 1, wherein the method to use the potentiometric urea sensor includes the following steps:
using an instrumentation amplifier as the readout circuit of the potentiometric urea sensor;
placing and stabilizing the potentiometric urea sensor into a buffer solution before measurement, and using the stabilized response potential as the reference potential;
placing the potentiometric urea sensor into a solution to be measured and recording the response potential.
